(19) United States
(12) Patent Application Publication
(10) Pub. No.: US 2011/0300574 A1
(43) Pub. Date: Dec. 8, 2011

(54) CELL-COUNTING METHOD FOR BLOOD HEMOLYSIS ANALYSIS IN FRAGILITY MEASUREMENT

(76) Inventors: Michael Tarasev, Pinkney, MI (US); Kenneth Alfano, Canton, MI (US)

(21) Appl. No.: 13/213,576

(22) Filed: Aug. 19, 2011

Related U.S. Application Data
(63) Continuation-in-part of application No. 12/690,916, filed on Jan. 20, 2010, now Pat. No. 8,026,102.
(60) Provisional application No. 61/146,145, filed on Jan. 21, 2009.

(51) Int. Cl. C12Q 1/02; G06K 9/00

(52) U.S. Cl. 435/29; 382/134

(57) ABSTRACT

A method of using cell counting to determine how much hemolysis occurs when a sample of red blood cells is subjected to stress, for purposes of either assessing stored blood quality and/or obtaining red blood cell fragility profiles. Cell-counting techniques and technologies can be employed to facilitate and enhance fragility measurements of red blood cells. There are many ways such “counting” can be achieved, including by visually counting each cell appearing under a microscope and projecting an estimated concentration based thereupon, or by utilizing a range of automatable technologies of various means. Using sophisticated image analysis algorithms, some such technologies also provide useful information about cell features or properties such as average size, distribution of sizes or volumes, and viability. Using cell counting to analyze post-stress hemolysis also permits conducting fragility assays without requiring centrifugation or other separation.

### CROSS-REFERENCE TO RELATED APPLICATIONS

This application is a continuation-in-part of application Ser. No. 12/690,916, filed Jan. 20, 2010, which claims the benefit of U.S. Provisional Application 61/146,145 filed Jan. 21, 2009, both of which are incorporated by reference in their entirety. The application also relates to U.S. Non-Provisional Utility application Ser. No. 11/744,643, filed May 4, 2007, which is incorporated by reference in its entirety.

### FIELD OF THE INVENTION

The present disclosure is in the technical field of medical apparatuses. More particularly, the present disclosure is in the technical field of quality control of stored red blood cell (RBC) units for the blood banking and transfusion industry.

### BACKGROUND OF THE INVENTION

This section contains general background material relevant to this continuation-in-part disclosure, which is not necessarily prior art.

Red blood cell (or erythrocyte) membrane fragility can be measured osmotically or mechanically; it involves subjecting a sample of cells to some physical stress and then measuring how much hemolysis occurred as a result of the stress. Fragility profiles can result when this is performed at multiple stress levels, and percentage hemolysis (or lysis) is plotted as the dependent variable thereof. Fragility values, indices or profiles for erythrocytes may be measured for research purposes or for clinical purposes, and the clinical applications can include the fields of blood-banking/transfusion medicine or diagnostic medicine.

### BRIEF SUMMARY OF THE INVENTION

This section briefly summarizes the subject matter of this continuation-in-part disclosure.

This present disclosure addresses a specific mode—cell counting—for determining how much hemolysis occurs when a sample of red blood cells is subjected to stress, as part of a fragility measurement, for purposes of either assessing RBC blood product quality and/or obtaining red blood cell fragility profiles (these two uses can overlap, such as when obtaining fragility profiles on stored blood). Note that while the testing itself is always performed in vitro, “blood product quality” can refer to either pre-transfusion or post-transfusion (e.g. drawn from the patient) blood samples, whereby the latter can refer to a test of cell properties' changes within a patient.

“Cell counting” techniques and technologies can be employed to facilitate and enhance certain fragility measurements of red blood cells. Fragility measurements require a step determining how much hemolysis occurs when a sample is subjected to stress; there are different ways this extent-of-hemolysis can be measured, some of which can involve counting cells to determine the concentration of unlysed cells both before and after subjecting them to stress(es) in order to compare. There are also many ways such “counting” can be achieved, including by manually/visually counting cells observed under a microscope and projecting an estimated concentration based upon that, or by utilizing a range of automatable technologies capable of doing so through various means.

Incorporating such approaches into red blood cell fragility measurement can provide a useful means (one that notably avoids the common need for centrifugation) for achieving the hemolysis measurement step in fragility assays targeted toward assessing stored blood quality, and/or in obtaining fragility profiles (in general), as well as enable additional useful information to be obtained which is not available otherwise. For example, some cell-counting technologies also provide information about cell features or properties such as average size, density, or distribution of sizes or volumes.

The scope of the invention is defined by the claims, which are incorporated into this section by reference. A more complete understanding of embodiments on the present disclosure will be afforded to those skilled in the art, as well as the realization of additional advantages thereof, by consideration of the following detailed description of one or more embodiments. Reference will be made to the appended sheets of drawings that will first be described briefly.

The following detailed description of the invention is merely exemplary in nature and is not intended to limit the invention or the application and uses of the invention. Furthermore, there is no intention to be bound by any theory presented in the preceding background of the invention or the following detailed description of the invention.

### BRIEF DESCRIPTION OF THE DRAWINGS

FIG. 1 is a flowchart showing an example sequence of a method for using cell-counting in a fragility assay to produce a red blood cell fragility profile.

FIG. 2 is a flowchart showing an example sequence of a method for using cell-counting in a fragility assay to assess quality of specific units of blood product.

### DETAILED DESCRIPTION OF THE INVENTION

Application Ser. No. 12/690,916 covers devices and methods for measuring red blood cell fragility in ways useful for assessing blood product quality, as well as devices and methods for multi-parameter measurement of red blood cell fragility of either blood product or patient blood (e.g. for diagnostic purposes). Those devices and methods essentially involve three main steps: 1) physically subjecting a sample of red blood cells to stress, 2) optically determining the relative fraction/proportion of hemolysis having occurred under said stress (directly or indirectly; explicitly or implicitly), and 3) processing the results of step 2 to characterize the red blood cells.

In application Ser. No. 12/690,916, the preferred specific optical mode of achieving step 2 was to spectrophotometrically ascertain the change in ratio of intracellular-to-extracellular hemoglobin caused by the stress inflicted during step 1; that particular mode of achieving step 2 is covered by U.S. Pat. No. 7,790,464. This present application addresses another specific mode (which can be either optical or non-optical) of achieving step 2 in such methods. Like the '464 approach to measuring hemolysis post-stress, this present approach to doing so is also a way to conduct that step without requiring centrifugation (which is typically needed in fragility measurements) to remove cells and fragments thereof from solution.

This present application addresses a specific mode—cell counting—for determining how much hemolysis occurs when a sample of red blood cells is subjected to stress, as part of a fragility measurement, for purposes of either assessing RBC blood product quality and/or obtaining red blood cell fragility profiles (these two uses can of course overlap, such as when obtaining fragility profiles on units of stored blood). Note that while the testing itself is performed in vitro, “blood product” quality can refer to either pre-transfusion or post-transfusion (e.g. drawn from the patient) blood samples, whereby the latter may regard a test of cell properties' changes having occurred within a patient; in such cases, the patient's own cardiovascular physiological stress might be the only stress employed in a given analysis. Blood product can also refer to red blood cells collected for a unit of product but not yet packaged as such.

“Cell counting” techniques and technologies can be employed to facilitate and enhance fragility measurements of red blood cells. Fragility measurements require determining how much hemolysis occurs when a sample is subjected to stress; there are different ways this extent-of-hemolysis can be measured, some of which include counting to determine the concentration of unlysed cells both before and after subjecting them to stress—in order to compare, and thereby also obtaining the amount of cells which did not survive the given stress. There are also many ways such counting can be achieved, including by manually/visually counting cells observed under a microscope and projecting an estimated concentration based upon that, or by utilizing a range of automatable technologies capable of doing so in various forms. Often a manual “eye” count (and thus any extrapolations based on the same) may be seemingly more precise, but this can be illusory as there is a higher error rate and smaller sample/subsample quantity counted. Of course, speed is another major difference. Using sophisticated image analysis algorithms or comparable capabilities, some such technologies also provide useful information about cell features or properties such as average size, distribution of sizes, or volumes.

Incorporating such approaches into red blood cell fragility measurement can provide useful means for accomplishing an important aspect and component of some fragility assays, and enable additional information to be obtained about either fragility values or fragility profiles. For example, identifying or associating particular cell diameter ranges with their respective hemolysis levels allows characterizing the properties of distinguishable cell subpopulations exhibiting varying propensities for lysis within a single given sample. Depending on whether the sample is taken from stored blood product or drawn from patient blood, such potential can then further be exploited to investigate patterns associated with particular types of blood product or particular patient conditions.

Cell-counting can be performed via light microscopy, either manually (“eye”) or via automated image-recognition systems and associated software. Many such systems are commercially available in a range of sophistication and automation (such as the multiple versions of the Cellometer™ product line, for example). When using brightfield light microscopy, cell staining may also be utilized—although this is typically not necessary for red blood cell counting. Alternatively, cell-labeling with fluorescent tags may employed to utilize fluorescence-based detection techniques for cell counting.

Note that non-fluorescent labeling, such as radio-labeling, may alternatively be employed for appropriate cell-counting applications. Use of such labeling would require corresponding detection instrumentality, such as when chromium labeling is used to conduct RBC post-transfusion survival studies (when validating blood product storage solutions).

Cell-counting can also be performed via electrical impedance or changes in resistance, most prominently by using Coulter counters. This approach is presently used for total cell counts in clinical laboratories. Certain models of Coulter counters also enable differentiation of cell subpopulations by size or volume, analogous to comparable features of the image-recognition systems associated with microscopy-based approaches. Recent versions are even small and hand-held, making them more conductive for increasing research and clinical applications.

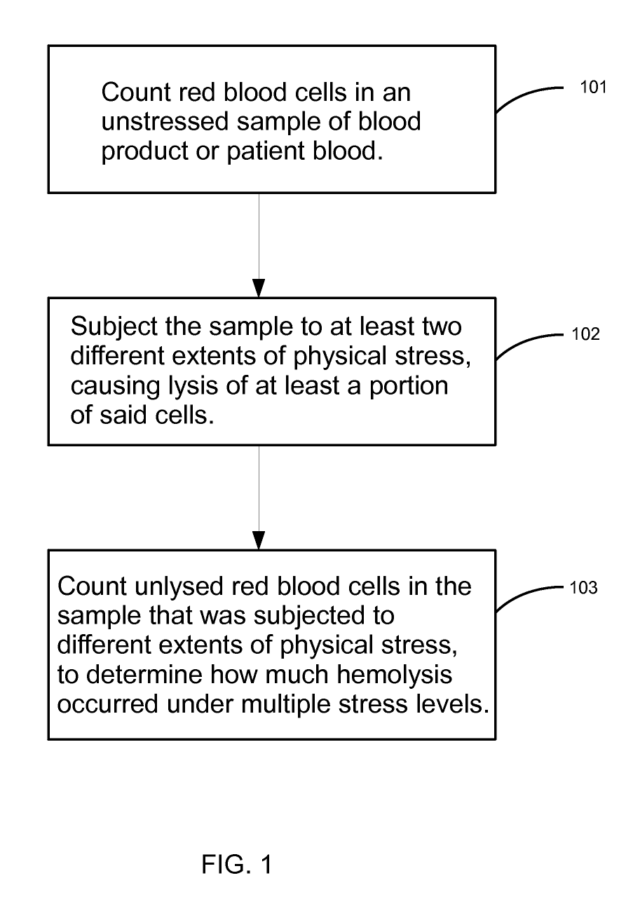

FIG. 1 is a flowchart showing an example sequence of a method for using cell-counting in a fragility assay to produce a red blood cell fragility profile. In step 101, count red blood cells in an unstressed sample of blood product or patient blood. Next in step 102, subject the sample to at least two different extents of physical stress, causing lysis of at least a portion of said cells. Finally in step 103, count unlysed red blood cells in the sample that was subjected to different extents of physical stress, to determine how much hemolysis occurred under multiple stress levels.

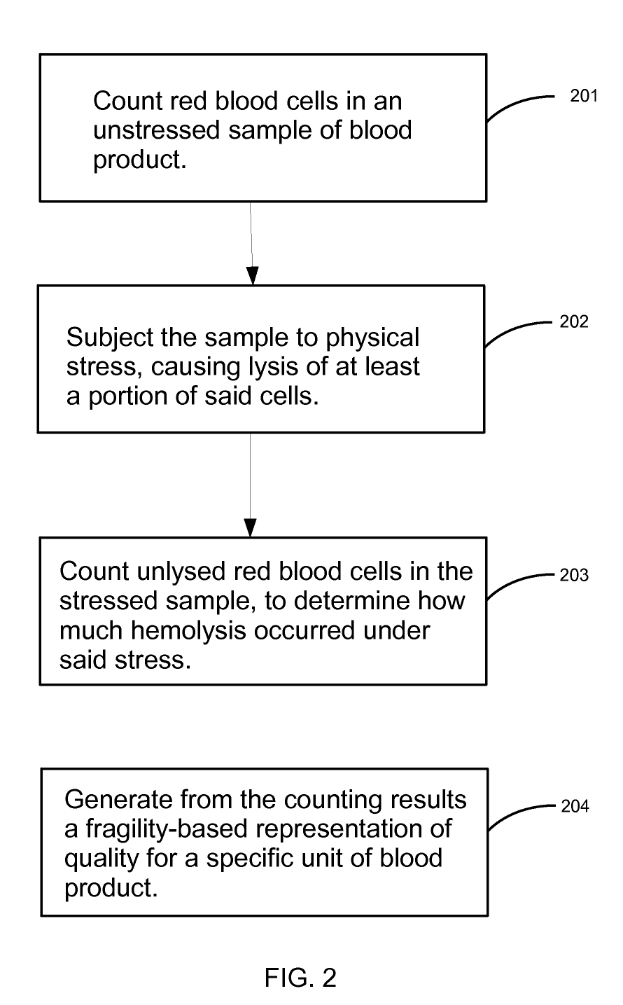

FIG. 2 is a flowchart showing an example sequence of a method for using cell-counting in a fragility assay to assess quality of specific units of blood product. In step 201, count red blood cells in an unstressed sample of blood product. Next in step 202, subject the sample to physical stress, causing lysis of at least a portion of said cells. Next in step 203, count unlysed red blood cells in the stressed sample, to determine how much hemolysis occurred under said stress. Finally in step 204, generate from the counting results a fragility-based representation of quality for a specific unit of blood product.

This disclosure enables those in the art to apply red-blood-cell-counting for obtaining fragility profiles thereof, as well as for obtaining fragility data thereof in evaluating quality of specific units of blood product. Moreover, those skilled in the art will appreciate readily apparent variations of the examples of principles described herein, which are also intended to be within the scope of the present invention.

We claim:

1. A method for analyzing hemolysis of blood product or patient blood to obtain data for a fragility profile having at least two data points, comprising:
counting red blood cells in a first sample of blood or blood product, when said sample has not been intentionally subjected to significant stress;
subjecting said first sample, or a second sample representative of the same source, to at least two different extents of physical stress, said stress causing lysis of at least a portion of said cells; and
counting unlysed red blood cells in the sample that was subjected to said different extents of physical stress, thereby determining an approximate extent of hemolysis occurring under at least two respective extents of physical stress.

2. The method of claim 1, wherein said physical stress is mechanical stress, and is applied in two or more distinct and variable parameters.

3. The method of claim 2, wherein said distinct and variable parameters comprise stress intensity and stress duration.

4. The method of claim 1, wherein said counting is performed utilizing light microscopy.

5. The method of claim 4, wherein said counting is performed utilizing fluorescence.

6. The method of claim 4, wherein said counting is performed utilizing an image analysis algorithm.

7. The method of claim 1, wherein said counting is performed utilizing electrical impedance or resistance or changes thereof.

8. The method of claim 1, further comprising determining an average cell size or a distribution of cell sizes within said blood product or patient blood.

9. The method of claim 8, wherein said hemolysis is determined for cell subpopulations, said subpopulations being characterized by different cell sizes within the stressed sample.

10. The method of claim 1, wherein said profile is obtained as part of a method for obtaining fragility-based quality information about specific units of blood product.

11. A method for analyzing hemolysis of blood product, as part of a method for obtaining fragility-based quality information about specific units of blood product, comprising:
counting red blood cells in a first sample of blood product, when said sample has not been intentionally subjected to significant stress;
subjecting said first sample, or a second sample representative of the same source, to physical stress, said stress causing lysis of at least a portion of said cells;
counting unlysed red blood cells in the sample that was subjected to said stress, to create results which indicate how much hemolysis occurs under said stress, said results comprising what fraction of cells did or did not lyse under given stress condition(s); and
generating from said results a representation of red blood cell quality for a specific unit of blood product, said representation of quality being based wholly or partially on red blood cell fragility data reflecting said results or subset(s) thereof.

12. The method of claim 11, wherein said stress is mechanical stress, and is applied in two or more distinct and variable parameters.

13. The method of claim 12, wherein said distinct and variable parameters comprise stress intensity and stress duration.

14. The method of claim 11, wherein said counting is performed utilizing light microscopy.

15. The method of claim 14, wherein said counting is performed utilizing fluorescence.

16. The method of claim 14, wherein said counting is performed utilizing an image analysis algorithm.

17. The method of claim 11, wherein said counting is performed utilizing electrical impedance or resistance or changes thereof.

18. The method of claim 11, further comprising determining an average cell size or a distribution of cell sizes within said blood product or patient blood.

19. The method of claim 18, wherein said hemolysis is determined for cell subpopulations, said subpopulations being characterized by different cell sizes within the stressed sample.

20. The method of claim 11, wherein said method utilizes a fragility profile, said profile comprising at least two data points representing an approximate fraction of said cells that had lysed under respective extents of said stress.
